(19) United States
(12) Patent Application Publication
(10) Pub. No.: US 2010/0213096 A1
(43) Pub. Date: Aug. 26, 2010

(54) Cyanoacrylate Adhesive Compositions and Devices and Process for Sterilization Thereof

(75) Inventor: Carlos R. Morales, Cumming, GA (US)

Correspondence Address: JONATHAN RIGDON SMITH , P.C, 1612 UNION ST, BRUNSWICK, GA, 31520-6733, US

(73) Assignee: CLAST TRADING LIMITED, Hong Kong (CN)

(21) Appl. No.: 12/633,795

(22) Filed: Dec. 8, 2009

Related U.S. Application Data
(63) Continuation-in-part of application No. 11/762,018, filed on Jun. 12, 2007, now abandoned.

(51) Int. Cl. B65D 85/00; A61L 2/04

(52) U.S. Cl. 206/524.1; 422/22

(57) ABSTRACT

The viscosity of 2-cyanoacrylate monomer based adhesives is increased by combining the monomers with suitable thickeners according to a certain process. The resulting formulations may be heat sterilized without degrading the viscosity or causing premature polymerization. The effectiveness of the sterilization process is assayed by disposing bacterial spores in the formulation, exposing it to a dry heat sterilization process, transferring it to a sterile aldose solution, transferring and exposing the sample to a nutrient medium which supports germination and growth of viable spores, incubating the samples, and determining the presence or absence of growth.

This application for patent is a continuation-in-part of U.S. application Ser. No. 11/762,018 filed on Jun. 12, 2007.

### DESCRIPTION

1. Field of Invention

Embodiments of the invention relate to compositions of cyanoacrylate monomer and polymer adhesive compositions for application in the medical and veterinary fields, processes for preparing such compositions, processes for sterilizing such compositions, and a method of assaying the sterilization of such compositions.

2. Background of Invention

It is known to use 2-cyanoacrylate esters as adhesives for bonding tissue in medical or surgical procedures performed upon the human or animal body. 2-cyanoacrylate esters polymerize rapidly, and often instantaneously, upon contact with tissue or body fluid. In these applications, the adhesive composition can be used to close wounds, as well as for covering and protecting surface injuries such as lacerations, abrasions, burns, sores and other open surface wounds. To be used in medical and veterinary fields, 2-cyanoacrylates must be sterilized. This is generally done in sealed containers to provide sterility, and from a practical perspective, to protect the compositions from moisture and premature polymerization. Previous sterilization methods involved either the use of ionizing radiation, including e-beam and gamma ray irradiation, dry heat at elevated temperatures (160° C.), or chemical sterilization such as with ethylene oxide.

When an adhesive composition is applied to a surface to be closed or protected, it is usually in its monomeric form, and the resultant polymerization produces the desired adhesive bond. However, at ordinary temperatures, the monomeric form of the adhesive has a low viscosity which results in the adhesive spreading into undesired areas. Therefore, it is desirable to increase the viscosity of the composition to prevent this unwanted flow. In order to achieve an increased viscosity, thickening agents can be added to the monomeric composition.

The previous methods of sterilization are undesirable in that the high temperatures required for the previous dry heat sterilization processes or irradiation could cause premature polymerization of the monomers. In addition, many polymers that could be used as thickeners underwent degradation resulting in loss of viscosity when treated with ionizing radiation or dry heat conditions of 160° C. This significantly limits the formulator's ability to formulate adhesive compositions which have the desirable stability and flow characteristics, and which can be sterilized.

### SUMMARY OF INVENTION

Embodiments of the present invention are directed to a method of sterilizing 2-cyanoacrylate compositions, including heating the composition in a device at a temperature of from about 90° C. to about 110° C. for an effective amount of time. In another aspect, embodiments of the invention include sterilized 2-cyanoacrylate ester compositions for use in medical applications or surgery, the compositions being disposed in sealed aluminum, tin, stainless steel, glass, or plastic containers and being sterilized at a temperature of between about 90° C. and about 110° C. In yet another aspect, embodiments of the invention are directed to a method for assaying the sterilization of cyanoacrylate compositions.

### DETAILED DESCRIPTION

Embodiments of the present invention provide a novel method of sterilizing 2-cyanoacrylate ester compositions using a dry heat means, and the resulting novel compositions. The combination of monomeric 2-cyanoacrylate, heat and time have a lethal effect on microbials, rendering sterilized compositions when the appropriate sterilization condition is achieved and when the method is applied to 2-cyanoacrylates in sealed containers.

As used herein, the following terms have the following meanings:

The term “cyanoacrylate adhesive composition” or “cyanoacrylate adhesive compositions” refers to polymerizable formulations comprising polymerizable cyanoacrylate ester monomers.

The term aldose is intended to refer to both common disaccharides and monosaccharides.

In a method of the invention, 2-cyanoacrylate adhesive compositions are sterilized through an unexpected and heretofore unknown combination of heat and time, sterilizing at temperatures significantly lower than previously thought to be effective. Previous dry heat sterilization methods have required temperatures of at least 160° C. to 180° C. Heating times at these temperatures were from two hours at 160° C. to 30 minutes at 180° C. Under the present invention, the 2-cyanoacrylate adhesive compositions can be sterilized at temperatures from about 90° C. to about 140° C. As would be expected, the time required to effect sterilization will vary depending on the temperature selected to accomplish the sterilization. At 140° C., sterilization requires approximately 30 minutes. At 70° C., sterilization requires about 600 minutes. Required heating times for intermediate temperatures are reported in Tables 2 and 3. Ultimately sterilization times for any composition can be readily determined by one skilled in the art by standard test methods without undue experimentation. Typical sterilization times are listed in Table 1.

TABLE 1
Sterilization heating times

| | |
|---|---|
| 70° C. | 600 minutes |
| 80° C. | 480 minutes |
| 90° C. | 300 minutes |
| 100° C. | 120 minutes |
| 110° C. | 90 minutes |
| 120° C. | 60 minutes |
| 130° C. | 60 minutes |
| 140° C. | 30 minutes |

According to an assay method of the invention, sterilization of cyanoacrylate compositions can be assayed for the effectiveness of a given temperature and sterilization time. Samples containing formulated n-butyl cyanoacrylate and 2-octyl cyanoacrylate in sealed borosilicate glass, aluminum tubes, and high density polyethylene (HDPE) containers were inoculated with Bacillus subtillis lyophilized spores at a concentration of 1×10⁶/ml of formulation. In other embodiments, spores can be introduced into the cyanoacrylate adhesive compositions prior to sterilization using commercially available biological indicators or spore test strips. Among the commercially available biological indicators which may be used are: bacterial spores on a stainless steel disc, bacterial spores on a steel wire, bacterial spores on steel coupons, bacterial spores on borosilicate paper and bacterial spores on woven cotton threads. Among the species of spores which may be chosen for use in the commercially available biological indicators are Bacillus subtillis and Geobacillus stearothermophilus. Commercially available biological indicators may be obtained from any commercial supplier, such as Raven Labs. Some inoculated glass vial and tube samples were kept at room temperature without sterilization as positive controls, while the rest of the samples were sterilized at temperatures ranging from 70 to 140° C. with different time exposures. Samples were sent to a microbiology laboratory for determination of the presence or absence of growth after the sterilization procedure was completed to assay the effectiveness of the process conditions.

In accordance with embodiments of the present invention it is preferred to utilize microorganisms which may be killed by the sterilization process but which show significant resistance to this process. The term microorganism refers to bacteria, fungi, yeast, protozoa algae, viruses and protozoa. Bacterial spores are very resistant to heat and chemicals, more so than vegetative bacterial cells, therefore the spores are often used to monitor sterilization procedures. A preferred organism for monitoring dry heat sterilization is Bacillus subtillis.

The spores represent a resting stage in the life cycle of the genus Bacillus. The resting spore contains a large number of active enzymes which allow the transformation from dormant cell to vegetative cell. The germination process, or the return to the vegetative state, has been described as a time-ordered sequence involving activation, triggering, initiation, and outgrowth. Activation is reversible and involves an increase in the rate and extent of germination. Triggering is irreversible and is the result of spore contact with the germinant. Initiation involves the loss of heat resistance, release of dipicolinic acid and calcium, loss of refractility, and absorbance. Outgrowth results in formation of the vegetative cell.

In accordance with embodiments of the present invention a cyanoacrylate composition test sample comprising at least one sterility test strip or lyophilized spores is utilized. While reference is made to “spores” as a test microorganism, it should be understood that microorganisms other than spore formers may be used in conjunction with the present invention. The spore strips utilized with the present invention are preferably constructed of materials which are inert to the microorganisms and inert to cyanoacrylate monomer. A variety of commercial spore strips is readily available and can be used with the present invention. The spore strips can contain more than one type of microorganism.

To assay the sterilized samples and controls, the compositions including the biological indicators are transferred into containers filled with an aqueous aldose solution, shaken, and transferred into a quantity of nutrient medium in an aseptic container. Transferring the samples to an aldose solution serves to emulsify the cyanoacrylate monomer without causing it to polymerize as it would upon exposure to water alone. Aldoses which act to emulsify the cyanoacrylate include without limitation, dextrose, lactose, arabinose, mannose, galactose, rhamnose, fructose, sucrose, and glucose. In one embodiment of the invention, the aldose is dextrose. The concentration of the aldose solution may be from about 2% to about 50% on a weight/weight basis. A preferred range for the concentration of the aldose solution is from about 3% to about 15%. A more preferred aldose concentration is from about 5% to about 10% weight/weight. The nutrient medium supports the germination of spores and growth of any viable microorganisms. The nutrient medium contains a protein substrate for the proteases liberated during spore germination and during subsequent microbial growth. The nutrient medium preferably comprises an aqueous solution or suspension of nutrient components (including the protein substrate) needed in order to promote the growth of viable microorganisms that may exist after the sterilization process. One example of a suitable culture medium is a protein containing microbiological broth such as tryptic soy broth (TSB) and/or TSB with specific protein additives, such as, for example, casein. Formulations for culture media are well known to those in the art.

The mixture of microorganisms, cyanoacrylate, aldose, and nutrient medium are sealed within a containing means. The samples are incubated for a predetermined period of time at from about 28° C. to about 37° C. Any microorganisms not killed during the sterilization process begin to germinate and grow during the incubation period. In a preferred embodiment the samples are incubated for at least about seven days. Thereafter the samples are examined to detect the presence of microorganism growth by different methods, such as visual examination of the samples followed by microscope Gram stain examination, addition of an enzymatic indicator such as tetrazolium salts followed by UV spectrophotometric analysis, or direct UV spectrophotometric analysis of incubated samples. In one embodiment, after visual examination a Gram stain smear is prepared to look for Gram positive rods which would confirm microorganism growth. In another embodiment, microorganism growth can be determined by the addition of an enzymatic biological indicator such as tetrazolium salts, wherein microorganism activity is determined by development of color which may be measured quantitatively with an ultraviolet spectrophotometer at 257 nm. In yet another embodiment, a sample without enzymatic indicator is analyzed under a spectrophotometer at a wavelength of 480 nm to determine microorganism growth.

A method of the invention can be applied in principle to any 2-cyanoacrylate ester monomer. The 2-cyanoacrylate is preferably an aliphatic cyanoacrylate ester and preferably an alkyl, cycloalkyl, alkenyl, alkoxyalkyl, fluoroalkyl, fluorocyclic alkyl or fluoroalkoxy-2-cyanoacrylate ester. The alkyl group may contain from 2 to 12 carbon atoms, and is preferably a C₂ to C₈ alkyl ester, and is most preferably a C₄ to C₈ alkyl ester. Suitable 2-cyanoacrylate esters include without limitation, the ethyl, n-propyl, iso-propyl, n-butyl, pentyl, hexyl, cyclohexyl, heptyl, n-octyl, 2-ethylhexyl, 2-methoxyethyl and 2-ethoxyethyl esters. Any of these 2-cyanoacrylate monomers may be used alone, or they may be used in mixtures.

The 2-cyanoacrylate monomers of the invention can be prepared by any of the methods known in the art. U.S. Pat. Nos. 2,721,858, 3,254,111, and 4,364,876, each of which is hereby incorporated herein in its entirety by reference, discloses methods for preparing 2-cyanoacrylates. For example, cyanoacrylates for the instant invention were prepared by reacting cyanoacetate with formaldehyde in the presence of heat and a basic condensation catalyst to give a low molecular weight polymer. A depolymerization step followed, under heat and vacuum in the presence of acidic and anionic inhibitors, yielding a crude monomer that could be distilled under vacuum and in the presence of radical and acidic inhibitors. The distilled 2-cyanoacrylate monomers were then formulated with radical and acidic inhibitors depending upon their application and to provide the necessary stability.

The 2-cyanoacrylate compositions of the invention may in some embodiments contain a thickening agent to increase the viscosity of the composition. This thickening agent may be a polymer. The thickening agent may be selected from the group consisting, without limitation, poly alkyl-2-cyanoacrylates, poly cycloalkyl-2-cyanoacrylates, poly fluoroalkyl-2-cyanoacrylates, of poly fluorocycloalkyl-2-cyanoacrylates, poly alkoxyalkyl-2-cyanoacrylates, poly alkoxycycloalkyl-2-cyanoacrylates, poly fluoroalkoxyalkyl-2-cyanoacrylates, polyalkoxycyclofluoroalkyl-2-cyanoacrylates, poly vinyl acetate, poly lactic acid and poly glycolic acid. In order to obtain optimum solubility of the polymer in the monomer, the polymer is often chosen to be a polymer of the monomer or one of the monomers which comprise the 2-cyanoacrylate composition. Preferably, the polymer is soluble in the monomer composition at ambient temperature. Preferred polymers include polymers of octyl 2-cyanoacrylate, vinyl acetate lactic acid, or glycolic acid. The preferred weight average molecular weight of the polymers is from about 300,000 to about 2,000,000. More preferably, the polymer molecular weight is from about 500,000 to about 1,600,000.

Cyanoacrylate polymers of the invention can be prepared by slow addition of the monomer to a mixer containing 0.1% bicarbonate deionized water. Water is then decanted away, and the polymer is rinsed several times with deionized water and decanted again. Following steps include neutralizing the polymer with 0.IN HCl, rinsing with deionized water, drying in a vacuum heated oven at a temperature of less than 80° C., and grinding the polymer to fine particles.

The amount of thickening agent that is added to the monomer composition is dependent upon the molecular weight of the polymer and the desired viscosity for the adhesive composition. The thickening agent typically is added at from about 1% to about 25% by weight of the composition. Preferably it is added at from about 1% to about 10%. More preferably it is added at from about 1% to about 5%. A typical viscosity of the composition is from about 25 to about 3000 centipoises, as measured by a Brookfield viscometer at 25° C. Preferably, the viscosity is between from about 50 to 600 centipoises at 25° C. The specific amount of a given thickening agent to be added can be determined by one of ordinary skill in the art without undue experimentation.

The 2-cyanoacrylate compositions may contain one or more acidic inhibitors in the range from 1 to 1,000 ppm. Such acidic inhibitors include without limitation: sulfur dioxide, nitrogen oxide, boron-oxide, phosphoric acid, ortho-, meta-, or para-phosphoric acid, acetic acid, benzoic acid, cyanoacetic acid, trifluoroacetic acid, tribromoacetic acid, trichloroacetic acid, boron trifluoride, hydrogen fluoride, perchloric acid, hydrochloric acid, hydrobromic acid, sulfonic acid, fluorosulfonic acid, chlorosulfonic acid, sulfuric acid, and toluenesulfonic acid.

The 2-cyanoacrylate compositions may contain one or more free radical polymerization inhibitors in the range from 0 to 10,000 ppm. Examples such radical inhibitors include, without limitation, catechol, hydroquinone, hydroquinone monomethyl ether, and hindered phenols such as butylated hydroxyanisol (BHA), butylated hydroxytoluene (2,6-di-tert-butyl butylphenol and 4-methoxyphenol), 4-ethoxyphenol, 3-methoxyphenol, 2-tert-butyl-4-methoxyphenol, and 2,2 methylene-bis-(4-methyl-6-tert-butyl phenol).

The 2-cyanoacrylate compositions may contain single or mixtures of plasticizers such as tributyl acetyl citrate, dimethyl sebacate, diethyl sebacate, triethyl phosphate, tri-(2-ethyl hexyl)phosphate, tricresyl phosphate, glyceryl triacetate, glyceryl tributyrate, dioctyl adipate, isopropyl myristate, butyl stearate, trioctyl trimellitate, and dioctyl glutarate. The plasticizers may be added to the compositions in proportions of less than 50% w/w of the formulation.

The 2-cyanoacrylate compositions may contain small amounts of dyes like the derivatives of anthracene and other complex structures. Some of these dyes include, without limitation, 1-hydroxy-4-[4-methylphenylamino]-9,10 anthracenedione (D&C violet No. 2), disodium salt of 6-hydroxy-5-[(4-sulfophenyl)axo]-2-naphthalene-sulfonic acid (FD&C Yellow No. 6), 9-(o-carboxyphenyl)-6-hydroxy-2,4,5,7-tetraiodo-3H-xanthen-3-one disodium salt monohydrate (FD&C Red No. 3), 2-(1,3 dihydro-3-oxo-5-sulfo-2H-indole-2-ylidine)-2,3-dihydro-3-oxo-1H-indole-5-sulfonic acid disodium salt (FD&C Blue No. 2), and [phthalocyaninato (2)] copper, added in proportions of less than 50,000 ppm.

The sterilized cyanoacrylate adhesive compositions of the invention may be packaged in a container made of any suitable material. Suitable materials must be heat stable and resistant up to the sterilization temperature, must provide an adequate barrier to atmospheric moisture and be compatible with the cyanoacrylate monomer or monomers. Materials meeting these requirements include metals, glass, and plastic. Suitable metals can include, without limitation, aluminum, tin, stainless steel, and plastics including high density polyethylene (HDPE), low density polyethylene (LDPE), polypropylenes, and phenolic resins. Especially useful plastics include surface-fluorinated plastics such as surface-fluorinated HDPE, surface-fluorinated LDPE, and surface-fluorinated polypropylene. Such plastics may be produced by the process of the Fluoro-Seal corporation (Fluoro-Seal, 16360 Park Ten Place, Suite 325, Houston, Tex. 77084-5046, www.fluoroseal.com) Metals can have different forms like pouches and tubes. Glass can be used as vials, breakable tubes or any other shape, and contained inside tubes made out of the same material, or combinations of materials including plastics. Particularly preferred materials are aluminum, type I borosilicate glass, and high density polyethylene (HDPE). Preferred aluminum tubes comprise a nozzle which is hermetically sealed by a pierceable membrane of aluminum, which are filled at their end remote from the nozzle prior to closure of the open end by tight crimping. The glass vials used in this invention are made out of type I borosilicate glass and sealed with a threaded phenolic cap with a silicone/Teflon® septum or sealed with an aluminum crimp cap and silicone/Teflon® septum. In the result, therefore, embodiments of the invention may reside in a substantially hermetically sealed aluminum container, e.g., an aluminum tube, containing a sterile 2-cyanoacrylate composition, or type I borosilicate glass vials hermetically sealed with a phenolic threaded cap and silicone/Teflon® septum, or in plastic containers made from HDPE, surface-fluorinated HDPE, LDPE, surface-fluorinated LDPE, polypropylene, surface-fluorinated polypropylene, phenolic resins, and the like.

### EXAMPLES

### Example I

### Sample Testing (Sterility Test Method for all Samples)

The method was tested by first performing the USP bacteriostasis and fungistasis test on glass vials and aluminum tubes. The sterility test was performed by obtaining spores of Bacillus subtillis var. niger suspended in irrigation water at a concentration of 2.3×10⁸/ml. Aliquots of 0.48 ml of these spores were placed in glass serum bottles, lyophilized and then reconstituted with 50 ml of n-butyl or 2-octylcyanoacrylate compositions to obtain a volume of 50 ml of inoculated spore solution with a concentration of 2×10⁶/ml. These cyanoacrylate spore solutions were used to fill the tubes and vials for the sterilization trials at different temperatures and times and for the non sterilized (standard biological indicators) control vials and tubes. Each tube and vial was filled with a volume of 0.5 to 0.6 ml of a cyanoacrylate composition that rendered a spore concentration of 2×10⁶/ml. Non sterilized biological indicator standards (BIs) and sterilized spore inoculated samples were transferred to a 5% dextrose USP solution, shaken, and transferred to soy casein digested broth (SCDB) and incubated at 35-37° C. for at least seven days. A vial of lyophilized spores with no cyanoacrylate was tested for population verification. The vial was transferred to sterile purified water and vortexed for 10 minutes. Serial dilutions of 10⁴, 10⁵, 10⁶ were plated in duplicate using SCDB and incubated for 48 hour at 35-37° C. The 10⁶ dilution yielded duplicate plates in the countable range. The final calculations showed there were 6.1×10⁶ CFU/ml, or 3.1×10⁷ CFU/vial.

Polymer Preparation: (Polymer Method for Samples Containing Polymer):

2-OCA polymer was made by adding drop-by-drop 30 grams of 2-OCA monomer to a blender containing 1000 ml of 0.1% sodium bicarbonate deionized water while swirling. Bicarbonate water with the polymer was vacuum filtered on a Kitasato with a Fisherbrand #Q5 quantitative filter paper, rinsed five times with 500 ml aliquots of deionized water, decanted and polymer neutralized with 500 ml of 0.1 N hydrochloric acid. The neutralized polymer was rinsed with three aliquots of 500 ml, decanted, dried in a vacuum oven at 80° C. and finely ground with a mixer after drying.

Sample Composition Preparation:

The sample of 2-OCA containing polymer was made by mixing 2-octyl cyanoacrylate (stabilized with 100 ppm of SO₂ and 1000 ppm of BHA) with 3.5% of 2-OCA polymer. The polymer was dissolved in the formulated 2-OCA by heating and mixing in a round glass flask equipped with a paddle shaft and mixer at a temperature no higher than 80° C. and obtaining a viscosity of 567 cp (measured with Brookfield DV-II at 25° C.). Then, the composition was inoculated with lyophilized Bacillus subtillis spores enough to produce a minimum concentration of 1×10⁶/ml which were deposited in aluminum tubes and type I borosilicate glass threaded vials. Tubes were sealed by crimping with a Kentex automatic tube filler and sealer. The glass vials were filled with an Eppendorf automatic pipette and sealed with threaded phenol caps and silicone/Teflon® septa.

Some inoculated glass and tube samples were not sterilized and were used as positive standard biological indicators to indicate viable spores. The rest of the inoculated and sealed tubes and vials were exposed to the experimental temperatures and time stipulated in the sterilization testing protocol conditions.

Tables 2 and 3 show example results.

TABLE 2
2-OCA sterilization example packed in glass vials
with pre-sterilization viscosity of 567 cp

| Sterilization | Sterilization | | Incubation | Number | Number of | Number | Viscosity @ |
|---|---|---|---|---|---|---|---|
| temperature | time | Type of | temperature | samples | days | of | 25° C. sterile |
| ° C. | minutes | media 400 ml | ° C. | tested | incubated | positives | cp |
| 90 | 240 | SCDB | 30-35 | 3 | 7 | 1 | 566 |
| 100 | 120 | SCDB | 30-35 | 3 | 7 | 0 | 569 |
| 100 | 180 | SCDB | 30-35 | 3 | 7 | 0 | 562 |
| 110 | 60 | SCDB | 30-35 | 3 | 7 | 0 | 526 |
| 110 | 120 | SCDB | 30-35 | 3 | 7 | 0 | 452 |
| 120 | 60 | SCDB | 30-35 | 3 | 7 | 0 | 418 |
| 120 | 90 | SCDB | 30-35 | 3 | 7 | 0 | N/A |
| 130 | 60 | SCDB | 30-35 | 3 | 7 | 0 | 343 |
| 130 | 120 | SCDB | 30-35 | 3 | 7 | 0 | N/A |
| 140 | 30 | SCDB | 30-35 | 3 | 7 | 0 | 110 |
| 140 | 45 | SCDB | 30-35 | 3 | 7 | 0 | N/A |

Table 2 above shows minimum sterilization temperatures, incubation temperature, incubation time and the results obtained for samples of Bacillus subtillis spores inoculated 2-OCA containing 3.5% 2-OCA polymer (567 cp), 100 ppm SO₂ and 1000 ppm BHA, packed in glass vials.

TABLE 3
2-OCA sterilization example packed in aluminum tubes
with pre-sterilization viscosity of 567 cp

| Sterilization | Sterilization | Type of | Incubation | Number | Number of | Number | |
|---|---|---|---|---|---|---|---|
| temperature | time | media | temperature | samples | days | of | Viscosity |
| ° C. | minutes | 400 ml | ° C. | tested | incubated | positives | @25° C. sterile |
| 90 | 240 | SCDB | 30-35 | 3 | 7 | 2 | 565 |
| 100 | 120 | SCDB | 30-35 | 3 | 7 | 0 | 566 |
| 100 | 180 | SCDB | 30-35 | 3 | 7 | 0 | 570 |
| 110 | 60 | SCDB | 30-35 | 3 | 7 | 0 | 526 |
| 110 | 120 | SCDB | 30-35 | 3 | 7 | 0 | 435 |
| 120 | 60 | SCDB | 30-35 | 3 | 7 | 0 | 405 |
| 120 | 90 | SCDB | 30-35 | 3 | 7 | 0 | N/A |
| 130 | 60 | SCDB | 30-35 | 3 | 7 | 0 | 351 |
| 130 | 120 | SCDB | 30-35 | 3 | 7 | 0 | N/A |
| 140 | 30 | SCDB | 30-35 | 3 | 7 | 0 | 102 |
| 140 | 45 | SCDB | 30-35 | 3 | 7 | 0 | N/A |

Table 3 above shows minimum sterilization temperatures, incubation temperature, incubation time and the results obtained for samples of Bacillus subtillis spores inoculated 2-OCA containing 3.5% 2-OCA polymer (567 cp), 100 ppm SO₂ and 1000 ppm BHA, packed in aluminum tubes.

Note the sharp drop in the viscosities of the compositions tested and shown in Tables 2 and 3 as temperature passes 110° C. The average viscosity drop from the base viscosity (567 cp) in the last column in each table going from row 4 to row 5 is 14.45%.

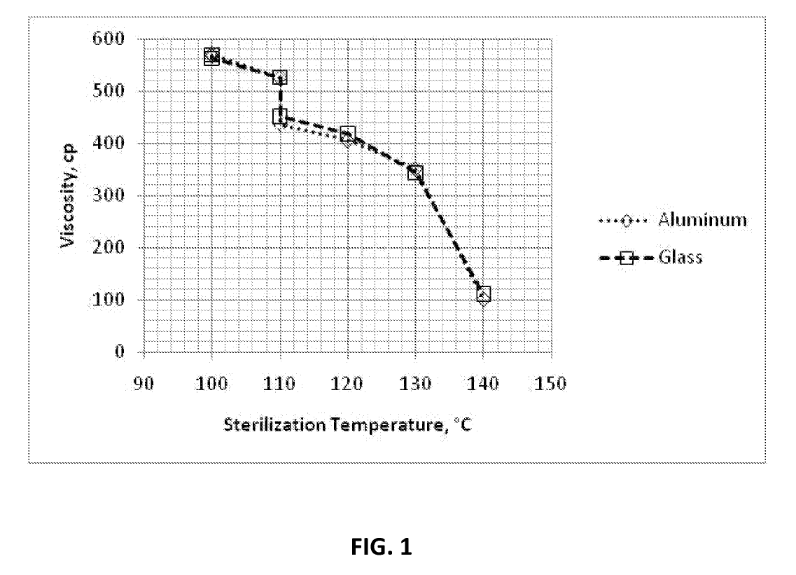

This is better illustrated in FIG. 1, a graph depicting the viscosity of the composition versus sterilization temperature for each of the two tested package materials (Table 2 and 3 data plotted together). The plotted data only include results in which the test for viable spores was negative after sterilization. There is a clear indication that the viscosity of the composition degrades sharply when the sterilization temperature exceeds about 100° C. The degradation curves for aluminum and glass packaging are virtually identical.

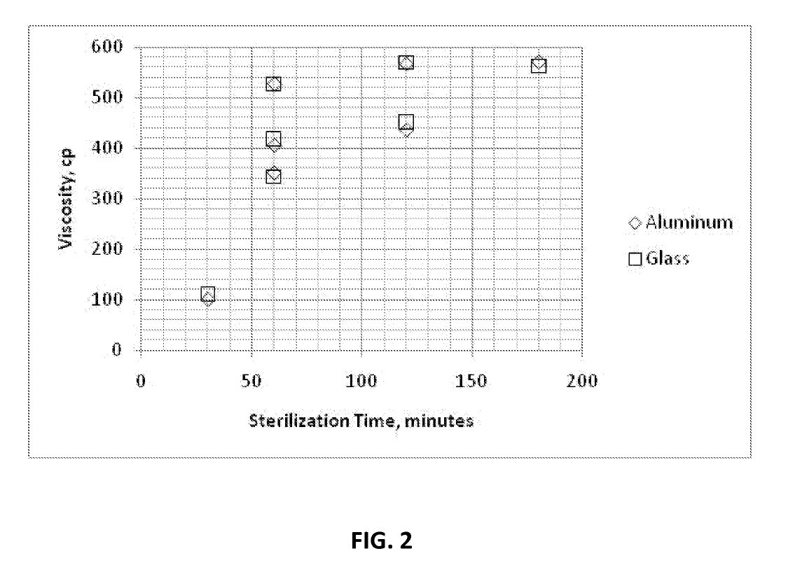

FIG. 2 depicts viscosity versus time for the same samples. Again, only sterilization times were plotted in which sterilization was complete. This graph shows a poorer correlation between viscosity and sterilization time; even samples held a long time retained their viscosity provided the temperature was held down.

### Example II

### Sample Composition Preparation

Sample II A:

A sample of n-butyl cyanoacrylate (n-BCA) with a viscosity of 2.8 cp (measured with a Brookfield DV-II at 25° C.) containing 100 ppm of SO₂ and 1000 ppm of butylated hydroxyanisole (BHA) was prepared for this example. Then, the composition was inoculated with BIs such as borosilicate spore discs, cotton threads and spore wires with a spore concentration of 1×10⁶/ml Geobacillus stearothermophilus. The spore-inoculated composition was deposited in type I borosilicate glass threaded vials with an Eppendorf automatic pipette and sealed with threaded phenol caps with silicone/Teflon® septa. Some inoculated vials were not sterilized and were used as positive standard biological indicators to indicate viable spores. The rest of the inoculated sealed vials were exposed to the experimental temperatures and times stipulated in the sterilization testing protocol conditions.

Table 4 shows example results.

TABLE 4
n-BCA monomer sterilization example, sterilization at 100° C.
in glass vials with pre-sterilization viscosity of 2.8 cp

| | Sterilization | Type of | Incubation | | | | |
|---|---|---|---|---|---|---|---|
| | time | media | temperature | Number | Number of | Number | Viscosity |
| Inoculant | minutes | 400 ml | ° C. | sample tested | days incubated | of positives | @25° C. sterile |
| Borosilicate disc | 240 | SCDB | 55-60 | 3 | 7 | 0 | 2.9 |
| Cotton threads | 240 | SCDB | 55-60 | 3 | 7 | 0 | 2.8 |
| SS wires | 240 | SCDB | 55-60 | 3 | 7 | 0 | 2.8 |
| Positive control | None | SCDB | 55-60 | 3 | 2 | 3 | 2.8 |
| borosilicate disc | | | | | | | |
| Positive control | None | SCDB | 55-60 | 3 | 2 | 3 | 2.9 |
| cotton threads | | | | | | | |
| SS wires | None | SCDB | 55-60 | 3 | 2 | 3 | 2.8 |

Table 4 above shows sterilization temperatures, incubation temperature, incubation time and the results obtained for samples of Geobacillus stearothermophilus spores inoculated n-BCA containing, 100 ppm SO₂ and 1000 ppm BHA.

Sample II B:

A sample of n-butyl cyanoacrylate (n-BCA) with a viscosity of 2.8 cp (measured with a Brookfield DV-II at 25° C.) containing 100 ppm of SO₂ and 1000 ppm of BHA was prepared for this example. Then, the composition was inoculated with cotton thread BIs with a spore concentration of 1×10⁶/ml Bacillus subtillis. The spore-inoculated composition was deposited in type I borosilicate glass threaded vials with an Eppendorf automatic pipette and sealed with threaded phenolic caps with silicone/Teflon® septa. Some inoculated vials were not sterilized and were used as positive standard biological indicators to indicate viable spores. The rest of the inoculated sealed vials were exposed to the experimental temperatures and times stipulated in the sterilization testing protocol conditions.

Table 5 shows example results.

TABLE 5
n-BCA monomer sterilization example at 100° C. in
glass vials with pre-sterilization viscosity of 2.8 cp

| | Sterilization | Type of | Incubation | Number of | Number of | | |
|---|---|---|---|---|---|---|---|
| | time | media | temperature | samples | days | Number of | Viscosity@25° C. |
| Inoculant | minutes | 400 ml | ° C. | tested | incubated | positives | sterile |
| Cotton | 240 | SCDB | 55-60 | 3 | 7 | 1 | 2.8 |
| threads | | | | | | | |
| Positive | None | SCDB | 55-60 | 3 | 2 | 3 | 2.8 |
| control | | | | | | | |
| cotton | | | | | | | |
| threads | | | | | | | |

Table 5 above shows sterilization temperatures, incubation temperature, incubation time and the results obtained for samples of Bacillus subtillis spores inoculated n-BCA containing 100 ppm SO₂ and 1000 ppm BHA.

### Example III

Samples of polymer modified 2-octyl (2-OCA) and n-butyl (n-BCA) cyanoacrylates stabilized with 100 ppm of SO₂ and 1000 ppm of BHA were filled and sealed in plastic HDPE pipettes with cotton thread BIs added with a spore population of 1×10⁶/ml. All samples were filled using an Eppendorf automatic pipette. Two samples were not sterilized and were used as positive controls.

TABLE 6
Samples containing a thick formulated mixture of 2-octyl and butyl
cyanoacrylate monomers with Bacillus subtillis biological cotton
thread indicators packed in plastic HDPE pipettes.

| | | | | | Number of | | |
|---|---|---|---|---|---|---|---|
| Sterilization | Sterilization | | Incubation | Number of | positive | Number of | |
| temperature | time | Type of | temperature | samples | controls | days | Number of |
| ° C. | minutes | media 400 ml | ° C. | tested | tested | incubated | positives |
| 110 | 180 | SCDB | 30-35 | 15 | N/A | 7 | 0 |
| | | | | Lot# | | | |
| | | | | 06L1201 | | | |
| 110 | 180 | SCDB | 30-35 | 15 | N/A | 7 | 0 |
| | | | | Lot# | | | |
| | | | | 06L0601 | | | |
| No | 0 | SCDB | 30-35 | N/A | 2 | 7 | 2 |

Table 6 above shows samples with a sterilization temperature of 110 C, incubation temperature, incubation time and the results obtained for samples and controls of formulated thick 2-OCA/nBCA, 100 ppm SO₂ and 1000 ppm BHA.

1. A method of sterilization of 2-cyanoacrylate adhesive composition, comprising:
heating the composition in a sealed container in a temperature range no lower than necessary to sterilize the composition in a selected amount of time, the temperature range also being no greater than necessary to
a) preserve the heat stability and heat resistance of the container, and
b) prevent more than a selected acceptable reduction in viscosity of the composition in the selected time;
the container having inner surface walls which are subjected to a means for treatment which, within the certain temperature range, provides an adequate barrier to atmospheric moisture and renders the walls compatible with the composition.

2. A method according to claim 1, wherein:
said certain temperature range is from about 100° C. to about 110° C.

3. A method according to claim 1, wherein:
said selected time is no less than about 90 minutes.

4. A method according to claim 1, wherein:
said means for treatment is selected from the group consisting of
a) surface-fluorination of HDPE,
b) surface-fluorination of LDPE, and
c) surface-fluorination of polypropylene.

5. A method according to claim 1, wherein:
said means for treatment is surface fluorination of HDPE.

6. A method as in claim 1, wherein:
the composition comprises one or more 2-cyanoacrylate ester monomers selected from the list of
a) alkyl2-cyanoacrylate monomer,
b) cycloalkyl 2-cyanoacrylate monomer,
c) fluoroalkyl 2-cyanoacrylate monomer,
d) fluorocycloalkyl 2-cyanoacrylate monomer, and
e) fluoroalkoxy 2-cyanoacrylate monomer.

7. A method according to claim 3, wherein:
the composition further comprises at least one thickener selected from the group consisting of
a) poly alkyl-2-cyanoacrylates,
b) poly cycloalkyl-2-cyanoacrylates,
c) poly fluoroalkyl-2-cyanoacrylates,
d) poly fluorocycloalkyl-2-cyanoacrylates,
e) poly alkoxyalkyl-2-cyanoacrylates,
f) poly alkoxycycloalkyl-2-cyanoacrylates,
g) poly fluoroalkoxyalkyl-2-cyanoacrylates,
h) poly alkoxycyclofluoroalkyl-2-cyanoacrylates,
i) poly lactic acid, and
j) poly glycolic acid.

8. A method according to claim 7, wherein:
said at least one thickener is a poly alkyl-2-cyanoacrylate.

9. A method according to claim 8, wherein:
the alkyl group of said poly alkyl-2-cyanoacrylate is selected from the group consisting of
a) straight chain C₄ to C₈ hydrocarbons and
b) branched chain C₄ to C₈ hydrocarbons.

10. A method according to claim 8, wherein:
said at least one thickener is poly octyl-2-cyanoacrylate.

11. A method according to claim 9, wherein:
said composition further comprises one or more anionic polymerization inhibitors plus one or more free radical polymerization inhibitors.

12. A method according to claim 1 wherein:
said selected acceptable reduction in viscosity is about 15%.

13. A method according to claim 7, wherein:
said composition further comprises a plasticizer selected from the group consisting of
a) tributyl acetyl citrate,
b) dimethyl sebacate,
c) diethyl sebacate,
d) triethyl phosphate,
e) tri-(2-ethylhexyl) phosphate,
f) tricresyl phosphate,
g) glycerol triacetate,
h) glycerol tributyrate,
i) dioctyl adipate,
j) isopropyl myristate,
k) butyl stearate
l) trioctyl trimellitate, and
m) dioctyl glutarate.

14. A method according to claim 13, wherein:
said plasticizer is up to 50% by weight of the cyanoacrylate composition.

15. An article of manufacture comprising:
a) a sealed container comprised of plastic, and
b) a composition comprising at least one 2-cyanoacrylate monomer and at least one thickener composed of at least one polymer made from the same at least one 2-cyanoacrylate monomer, the at least one thickener prepared and added to the at least one 2-cyanoacrylate monomer by a thickener preparation and addition process, sterilized by heating to a temperature no greater than about 110° C. for a period of time sufficient to sterilize the composition.

16. The article of manufacture of claim 15, wherein:
said thickener preparation and addition process comprises the steps of
a) selecting a polymer to use as said thickener that is compatible with said at least one 2-cyanoacrylate monomer;
b) adding the polymer to a mixer containing 0.1% bicarbonate deionized water;
c) rinsing the polymer several times with deionized water, each time decanting the water;
d) neutralizing the polymer with 0.1N HCl and rinsing with deionized water;
e) drying the polymer in a vacuum heated oven at a temperature no greater than about 80° C.;
f) grinding the polymer to fine particles; and
g) adding the polymer to the at least one 2-cyanoacrylate monomer in an amount that is no more than the solubility limit of the polymer in the at least one 2-cyanoacrylate monomer at about 20° C.

17. The article of manufacture according to claim 15, wherein:
said period of time is no less than about 90 minutes.

18. The article of manufacture according to claim 15, wherein:
said plastic container made from a plastic selected from the group consisting of
a) HDPE,
b) surface-fluorinated HDPE,
c) LDPE,
d) surface-fluorinated LDPE,
e) polypropylene,
f) surface-fluorinated polypropylene, and
g) phenolic resin.

19. The article of manufacture according to claim 18, wherein:
said plastic is surface-fluorinated HDPE.

20. The article of manufacture according to claim 15, wherein:
said composition comprises one or more 2-cyanoacrylate ester monomers taken from the group consisting of
a) alkyl 2-cyanoacrylate ester monomers,
b) cycloalkyl 2-cyanoacrylate ester monomers,
c) fluoroalkyl 2-cyanoacrylate ester monomers,
d) fluorocycloalkyl 2-cyanoacrylate ester monomers, and
e) fluoroalkoxy 2-cyanoacrylate monomers.

21. The article of manufacture according to claim 15, wherein:
said composition further comprises at least one thickener selected from the group consisting of
a) poly alkyl-2-cyanoacrylates,
b) poly cycloalkyl-2-cyanoacrylates,
c) poly fluoroalkyl-2-cyanoacrylates,
d) poly fluorocycloalkyl-2-cyanoacrylates,
e) poly alkoxyalkyl-2-cyanoacrylates,
f) poly alkoxycycloalkyl-2-cyanoacrylates,
g) poly fluoroalkoxyalkyl-2-cyanoacrylates,
h) poly alkoxycyclofluoroalkyl-2-cyanoacrylates,
i) poly lactic acid, and
j) poly glycolic acid.

22. The article of manufacture according to claim 21, wherein:
said at least one thickener is at least one poly alkyl-2-cyanoacrylate.

23. The article of manufacture according to claim 22, wherein:
the alkyl group of said at least one poly alkyl-2-cyanoacrylate is selected from the group consisting of
a) straight chain C₄ to C₈ hydrocarbons, and
b) branched chain C₄ to C₈ hydrocarbons.

24. The article of manufacture according to claim 21, wherein:
said at least one thickener is poly octyl-2-cyanoacrylate.

25. The article of manufacture according to claim 15, wherein:
said composition consists of a stabilizer taken from the group consisting of
a) one or more of an anionic polymerization inhibitor,
b) one or more of a free radical polymerization inhibitor, and
c) the combination of one or more anionic polymerization inhibitors and one or more free radical polymerization inhibitors.

26. The article of manufacture according to claim 15, wherein:
said cyanoacrylate composition further comprises a plasticizer selected from the group consisting of
a) tributyl acetyl citrate,
b) dimethyl sebacate,
c) diethyl sebacate,
d) triethyl phosphate,
e) tri-(2-ethylhexyl) phosphate,
f) tricresyl phosphate,
g) glycerol triacetate,
h) glycerol tributyrate,
i) dioctyl adipate,
j) isopropyl myristate,
k) butyl stearate
l) trioctyl trimellitate, and
m) dioctyl glutarate.

27. The article of manufacture according to claim 26, wherein:
said plasticizer is up to 50% by weight of the composition.

28. An article of manufacture, comprising:
a sterile composition comprising of one or more 2-cyanoacrylate monomers for use in medicine or surgery;
the composition disposed in a sealed container comprised of surface-fluorinated HDPE;
the composition sterilized by heating to a temperature no greater than about 110° C. for a period of time sufficient to sterilize the composition;
the composition further comprising
a thickener comprised of 2-cyanoacrylate polymers made from the identical one or more monomers and prepared and added to the one or more monomers by a thickener preparation and addition process;
the composition further comprising
a stabilizer comprised of at least one anionic polymerization inhibitor and at least one free radical polymerization inhibitor; and
the composition further comprising
a plasticizer wherein
the plasticizer is up to 50% by weight of the composition.

29. A method of preparing, sterilizing, and maintaining the stability of a 2-cyanoacrylate adhesive composition, comprising the steps of:
a) dissolving approximately 20 to 1000 ppm by weight of SO₂ and approximately 20 to 10,000 ppm by weight of butylated hydroxyanisole (BHA) in a liquid consisting of one or more 2-cyanoacrylate ester monomers;
b) dissolving a thickener prepared according to the method of claim 16 in the resulting mixture;
c) dissolving up to approximately 50% by weight of plasticizer in the resulting liquid;
d) placing the resulting liquid a container having an inner surface that is fluorinated;
e) sealing the container with a seal that is heat stable and heat resistant up to the sterilization temperature, provides an adequate barrier to atmospheric moisture, and is compatible with the composition; and
f) heating said composition in the container at a temperature that is
no lower than necessary to sterilize the composition in a selected time, and
no greater than that at which the container is heat stable and heat resistant, at which the container provides an adequate barrier to atmospheric moisture, at which the container is compatible with the composition, and at which no more than an acceptable loss in viscosity occurs, whichever is lower.

30. The method of claim 29, wherein:
said thickener has an initial molecular weight in the range of from about 300,000 to about 2,000,000 daltons.

31. The method of claim 29, wherein:
said thickener has an initial molecular weight in the range of from about 500,000 to about 1,600,000 daltons.

32. The method of claim 29, wherein:
said thickener is added in the range of from about 1% to about 25% by weight of the composition.

33. The method of claim 29, wherein:
said thickener is added in the range of from about 1% to about 10% by weight of the composition.

34. The method of claim 29, wherein:
said thickener is added in the range of from about 1% to about 5% by weight of the composition.

35. The method of claim 29, wherein:
said thickener is added to produce a viscosity of the composition from about 25 to about 3000 centipoises.

36. The method of claim 29, wherein:
said thickener is added to produce a viscosity of the composition from about 50 to about 600 centipoises.

37. The method of claim 29, wherein:
said temperature no lower than necessary to sterilize the composition is about 100° C.; and
said temperature no greater than that which causes more than an acceptable loss in viscosity is greater than about 110° C.

38. The method of claim 29, wherein:
said selected time is no less than about 90 minutes.

39. The method of claim 29, wherein:
said acceptable loss in viscosity is no greater than about 15%.

40. A composition according to claim 25, wherein:
said combination of one or more anionic polymerization inhibitors and one or more free radical polymerization inhibitors is SO₂ and BHA.
